(19) United States
(12) Patent Application Publication
(10) Pub. No.: US 2004/0213738 A1
(43) Pub. Date: Oct. 28, 2004

(54) CIRL3-Like proteins, nucleic acids, and methods of modulating CIRL3-L-mediated activity

(76) Inventors: Susan Croll-Kalish, Tarrytown, NY (US); Richard Torres, New York, NY (US); Andrew J. Murphy, Croton-on-Hudson, NY (US)

Correspondence Address: REGENERON PHARMACEUTICALS, INC, 777 OLD SAW MILL RIVER ROAD, TARRYTOWN, NY, 10591, US

(21) Appl. No.: 10/804,532

(22) Filed: Mar. 19, 2004

Related U.S. Application Data
(60) Provisional application No. 60/459,076, filed on Mar. 31, 2003.

(51) Int. Cl. A61K 51/00; C12Q 1/68; G01N 33/53; G01N 33/567

(52) U.S. Cl. 424/1.49; 435/6; 435/7.2

(57) ABSTRACT

Provided is a human Calcium Independent Receptor of Latrotoxin 3-Like (CIRL3-L) protein, as well as the encoding nucleic acid, methods for screening for agents capable of modulating CIRL3-L related activity and treating CIRL3-L-mediated conditions. Further provided are animal models useful for screening agents capable of ameliorating or reducing anxiety related disorders, obsessive-compulsive disorders, seizure related disorders and autism and other pervasive developmental disorders.

### CROSS-REFERENCE TO RELATED APPLICATIONS

This application claims the benefit under 35 USC § 119(e) of U.S. Provisional 60/459,076 filed 31 Mar. 2003, which application is herein specifically incorporated by reference in its entirety.

### BACKGROUND OF THE INVENTION

1. Field of the Invention

This invention is related to methods for identifying molecules capable of modulating CIRL3-Like (CIRL3-L) protein, therapeutic uses for such identified molecules, and animal models of human psychiatric disorders and seizure-related disorders.

2. Description of Related Art

G-protein coupled receptors (GPCRs) are a class of integral membrane proteins, which contain seven hydrophobic transmembrane domains that span the cell membrane and form a cluster of anti-parallel alpha helices. GPCRs function in various physiological processes including vision, smell, neurotransmission, and hormonal responses. Calcium Independent Receptor of Latrotoxin 3. Like (CIRL3-L) was originally identified as a homologue of CIRL3, a homologue of CIRL1.

Current treatments for anxiety disorders include tricyclic antidepressants (TCAs), selective serotonin reuptake inhibitors (SSRIs), and classical irreversible monoamine oxidase inhibitors (MAOIs). These are commonly used in the treatment in a broad range of anxiety d disorders, including Generalized Anxiety Disorder (GAD) and Obsessive Compulsive Disorder (OCD). However, the poor tolerance of TCAs and the cardiac risks associated therewith, as well as the risks associated with conventional irreversible MAOIs, are limitations to their usefulness. Additionally, SSRIs have a slow onset of action, and are effective in less than two-thirds of patients.

### BRIEF SUMMARY OF THE INVENTION

A new GPCR protein, designated Calcium Independent Receptor of Latrotoxin 3-Like (CIRL3-L) (SEQ ID NO:1) and the nucleic acid that encodes it (SEQ ID NO:2), are described herein. This protein has a role in the mediation of psychiatric disorders including anxiety disorders and schizophrenia, as well as nervous and compulsive motor activity. Additionally, CIRL3-L has a role in the mediation of seizures. The discovery of this protein allows for screening and therapeutic methods leading to the development of a novel therapeutics useful for modulating these activities.

Accordingly, in a first aspect, the invention provides a nucleotide sequence encoding human CIRL3-L, comprising the sequence of SEQ ID NO:2; nucleotide sequences which hybridize under stringent conditions to the SEQ ID NO:2; nucleotide sequences having 90%, 95%, 98% and 99% homology to SEQ ID NO:2; and nucleotide sequences varying from the sequence of SEQ ID NO:2 as a result of degeneracy of the genetic code.

In a second related aspect, the invention provides a protein having the sequence of SEQ ID NO:1, as well as protein sequences having identity to SEQ ID NO:1 of at least 95%, 97%, 98%, or 99%.

In a third aspect, screening methods are provided for identifying agents capable of binding a human CIRL3-L protein. More specifically, the invention provides methods of identifying agents capable of modulating (e.g., enhancing or inhibiting) human CIRL3-L-mediated activity. Such agents are valuable as potential therapeutics for the treatment of psychiatric and neurological disorders such as the anxiety disorders, such as, obssessive-compulsive disorder (OCD), the pervasive developmental disabilities (PDDs) such as Asperger's Syndrome, Autism, and pervasive developmental disabilities not otherwise specified (PDD-NOS) and schizophrenia. In addition, these agents may be useful as therapeutics in the treatment of seizures and related disorders. The screening methods of the invention include in vitro and in vivo assays.

In one embodiment of an in vitro screening method of the invention, agents capable of binding the CIRL3-L protein are identified in a cell-based assay system. More specifically, cells expressing a CIRL3-L protein are contacted with a test compound, and the ability of the test compound to bind CIRL3-L or a fragment thereof is determined.

In another specific embodiment of the cell-based assay of the invention, the ability of a test compound to bind to CIRL3-L may be determined by a competitive binding assay. Accordingly, the ability of the test compound to competitively bind to CIRL3-L may be determined by obtaining cells expressing CIRL3-L, contacting the cells with one agent known to bind to CIRL3-L and a second agent whose ability to bind CIRL3-L is unknown, detecting the amount of binding of the first agent and comparing that amount with the amount of binding of the second agent. Binding of a compound to CIRL3-L may be determined in a number of ways known to the art, including for example, radioactive detection, fluorescence detection, chromogenic detection, mass spectroscopy, and plasmon resonance, or by detection of a biological response through measurement of Ca²⁺ ion flux, cAMP, IP₃, PIP₃ and transcription of reporter genes.

In another embodiment, agents capable of binding a CIRL3-L protein are identified in a cell-free assay system. More specifically, a native or recombinant human CIRL3-L protein is contacted with a test compound, and the ability of the candidate compound to bind CIRL3-L is determined.

In another embodiment, agents capable of binding CIRL3-L or a fragment thereof are identified in an in vivo system. More specifically, a candidate agent or a control compound is administered to a suitable animal, and the effect on CIRL3-L-mediated activity is determined.

In a fourth aspect, screening methods are provided for identifying antagonists of the human CIRL3-L protein. The method of the invention includes in vitro screening assay, including cell-free and cell-based assays, as well as in vivo assays. More specifically, an antagonist of the human CIRL3-L protein is capable of inhibiting or blocking the activity and/or expression of human CIRL3-L. In a more specific embodiment, the agent capable of inhibiting CIRL3-L-mediated activity decreases the activity of human CIRL3-L, for example, a blocking antibody. In another more specific embodiment, the antagonist is capable of interfering with the expression of the gene encoding CIRL3-L, such as for example, an antisense or siRNA molecule. Generally, manipulation of CIRL3-L levels are believed to be therapeutically useful to alleviate obsessive compulsive disorders and the pervasive developmental disorders.

In a fifth aspect, screening methods are provided for identifying agonists of the human CIRL3-L protein. The method of the invention includes in vitro screening assay, including cell-free and cell-based assays, as well as in vivo assays. More specifically, an agonist of the human CIRL3-L protein is capable of enhancing the activity and/or expression of human CIRL3-L. In a more specific embodiment, the agent capable of stimulating CIRL3-L-mediated activity increases the activity of human CIRL3-L, such as for example, an activating antibody. In another more specific embodiment, the agonist is capable of increasing the expression of the gene encoding CIRL3-L. Generally, agonists of CIRL3-L is believed to be therapeutically useful in the treatment of autism or pervasive developmental disorders.

Agents identified by the method of the invention are potential therapeutics useful in the treatment of psychiatric, and some neurological disorders, such as, for example, anxiety, OCD, autism, PDD-NOS, Asperger's Syndrome, Tourette's Syndrome, and schizophrenia. Any suitable test known to the art for identifying and measuring psychiatric disorders in a test animal may be used to identify an agent useful in the treatment of psychiatric disorders in humans, such as the tests described below, e.g., the “elevated plus maze”, open field testing, light-dark exploration tests, social interaction testing, sensory testing, sensory gating testing, and/or quantification of animal freezing, defecations, rears and grooming (especially early or excessive).

Agents identified by the method of the invention are also potential therapeutics useful in the treatment of seizures and related disorders, such as, for example, generalized or partial seizures such as temporal lobe epilepsy, absence seizures, febrile seizures, juvenile myoclonic epilepsy, West syndrome, Lennox-Gastaut Syndrome, and Rasmussen's encephalitis. Any suitable test known to the art for treating seizures and related disorders in a test animal may be used to identify an agent useful in the treatment of seizures and related disorders in humans, such as kindling epileptogenesis, pilocarpine-induced seizures, pentylenetetrazol-induced seizures, kainic acid-induced seizures, flurothyl-induced seizures, and hilar lesion-induced seizures.

In a sixth aspect, the invention embodies therapeutic methods for treating a CIRL3-L-mediated condition, comprising administering an agent capable of modulating CIRL3-L activity identified by a screening method of the invention to a subject in need thereof. In the therapeutic method of the invention, a CIRL3-L-mediated condition is a psychiatric disorder, such as schizophrenia and anxiety disorders including generalized anxiety disorder (GAD) and obsessive-compulsive disorder (OCD), or neurological disorders characterized by compulsive or perseverative behavior such as autism, Asperger's Syndrome and Tourette's Syndrome, or disorders characterized by social impairments such as autism, Asperger's Syndrome, PDD-NOS, and schizophrenia. A CIRL3-L-mediated condition may result from neurological impairment, which may be congenital or the result of trauma. In one embodiment, the agent administered is a compound identified through a screening method of the invention.

In another method of the invention, a CIRL3-L-mediated condition is seizure and related disorders, such as, for example, generalized or partial seizures such as occur in temporal lobe epilepsy, absence seizures, febrile seizures, juvenile myoclonic epilepsy, West syndrome, Lennox-Gastaut Syndrome, Rasmussen's encephalitis, or as a consequence of trauma or damage to the brain, such as, for example, head trauma, stroke, brain tumors, or cerebrovascular abnormalities. The invention is applicable to populations particularly at risk for seizures and related disorders. Such populations may be identified by, and include, for example, a subject known to experience or suspected of being at risk for generalized or partial seizures such as occur in temporal lobe epilepsy, absence seizures, febrile seizures, juvenile myoclonic epilepsy, West syndrome, Lennox-Gastaut Syndrome, Rasmussen's encephalitis, developmental disorders, such as autism, PDDs, Asperger's Syndrome, cortical dysplasias, or Down's syndrome, or damage to the brain.

In seventh aspect, the invention features pharmaceutical compositions useful for treatment of CIRL3-L-mediated psychiatric disorders and diseases, for diminishing anxiety and anxiety-related activity, or for modulating CIRL3-L-mediated motor activity, comprising an agent identified by a screening method of the invention.

In an eighth aspect, the invention features pharmaceutical compositions useful for treatment of CIRL3-L-mediated seizure and related disorders, in a subject suffering from or at risk thereof, comprising an agent identified by the screening method of the invention.

In a ninth aspect, the invention features a non-human transgenic animal comprising a modification of an endogenous CIRL3-L gene. As described more fully in U.S. Pat. No. 6,856,251, the transgenic animal of the invention is generated by targeting the endogenous CIRL3-L gene with a large targeting vector (LTVEC). In one embodiment of the transgenic animal of the invention, the animal is a knock-out wherein the CIRL3-L gene is altered or deleted such that the function of the endogenous CIRL3-L protein is reduced or ablated. In another embodiment, the transgenic animal is a knock-in animal modified to comprise an exogenous human CIRL3-L gene. Such transgenic animals are useful, for example, in identifying agents that diminish anxiety or modulate other activities that are mediated by the human CIRL3-L protein. Such transgenic animals are also useful in identifying agents that treat seizures and related disorders mediated by the human CIRL3-L protein.

In a related tenth aspect, the invention provides an animal model for use in identifying an agent capable of diminishing, reducing, and/or ameliorating psychiatric or neurological disorders. The CIRL3-L gene knock-out animals of the invention exhibit specific symptoms of compulsive motor activity, perseverative behavior, abnormal social behavior, and anxiety, and are thus useful in a variety of ways, including in vivo screening of potential therapeutic compounds capable of ameliorating, diminishing, or reducing GAD, autism, PDD-NOS, Asperger's Syndrome, and/or OCD. The effectiveness of the test agent may be determined by behavioral observation, such as for example observation of an animal in a elevated plus maze, light-dark exploration task, “Y” maze, social interaction tests, sensory tests, sensory gating tests, or open field.

The animal model of the invention may also be used to screen for agents capable of treating of seizure and related disorders, by administering a test agent to the animal and determining the ability of the test agent to treat seizure and disorders associated therewith. The effectiveness of the test agent may be determined by seizure threshold, seizure severity, EEG amplitude or frequency changes, or histopathological evidence of seizure-related neural alterations including, but not limited to, neuronal cell death, mossy fiber sprouting, or gliosis.

Other objects and advantages will become apparent from a review of the ensuing detailed description.

### BRIEF DESCRIPTION OF THE FIGURES

FIGS. 1A-F are bar graphs showing social behavior parameters in CIRL3-L knockout (KO) and wildtype (wt) mice. All KO mice show social impairment, but the severity of the impairment depends upon the background strain of the mice. F2 mice (A-C) show willingness to initiate social contact, but inappropriate social behaviors such as following (B) and inappropriate contact (C). N2F2 mice, which are genetically more similar to C57B1/6 mice, show significant impairments in willingness to socialize (D), including a failure to initiate inappropriate behaviors (E and F).

FIGS. 2A-B are bar graphs showing gait abnormalities CIRL3-L KO and wt mice. CIRL3-L KO mice show mild, but significant, gait abnormalities, characterized by significantly decreased forepaw inter-step distance (A), and significantly reduced stride length (B).

FIGS. 3A-C are bar graphs showing nociceptive sensation in CIRL3-L KO and wt mice. CIRL3-L KO mice show impaired nociceptive responding on the hot plate test, which contains a central perceptual component (A), but not the tail flick test, which is a measure of reflexive spinal pain (B). The KOs also show impaired cutaneous sensation (C), suggestive of a more general somatosensory processing deficit.

FIGS. 4A-D are bar graphs showing anxiety in CIRL3-L KO and wt mice. CIRL3-L KO mice show significant evidence of compulsive motor anxiety, as demonstrated by increased number of grooming bouts (A) and decreased latency to groom (B). In contrast, they do not exhibit significant overt generalized anxiety as measured by the light-dark exploration task (C) or the elevated plus maze (D).

FIGS. 5A-B are bar graphs show in the effect of imipramine treatment on CIRL3-L KO and wt mice. CIRL3-L KO mice show some reductions in their compulsive grooming behaviors after 2 weeks treatment with imipramine. This reduction can be observed in both latency to groom (A) and number of grooms (B).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

Definitions

By the term “CIRL3-L” protein is meant a protein having the sequence of SEQ ID NO:1, or a functional equivalent thereof. By the term a “functional equivalent of CIRL3-L” is meant a protein that substantially has the function or the activity of CIRL3-L, and has at least 90%, preferably at least 95%, most preferably at least 99% homology in the nucleotide sequence encoding the protein or the amino acid sequence, when optimally aligned with the original CIRL3-L protein. Such a functional equivalent of CIRL3-L includes substitution, addition, deletion or insertion of at least one nucleotide, in addition to the original sequence in the terminal inverted repeat sequence or the open reading frame that is a functional site, and has at least functions or activities substantially equivalent to those of the original CIRL3-L protein. Such a functional equivalents may include substitution of at least one amino acid (preferably conservative substitution), or additional amino acid (e.g., a reader sequence, a secretion sequence, and a sequence that would advantageously function in purification), in addition to the original sequence. It is appreciated that production of these functional equivalents is within a scope of technical knowledge that can be routinely obtained by those skilled in the art.

By the term “CIRL3-L-mediated condition” is meant a disease or condition associated with activity and/or expression of the CIRL3-L protein. More specifically, and as shown in the experimental results below, the CIRL3-L knock-out animals of the invention show that CIRL3-L is localized to several important regions of the brain, particularly in the thalamus and anterior cingulate cortex. These sites indicate a role of CIRL3-1 in psychiatric disorders, such as anxiety and OCD. Moreover, these knock-out animals exhibit specific symptoms of compulsive motor activity, perseverative behavior, and anxiety, and may further exhibit increased incidence and/or risk of seizures or disorders associated therewith.

A “knock-out” animal is an animal generated from a mammalian cell that carries a genetic modification resulting from the insertion of a DNA construct targeted to a predetermined, specific chromosomal location that alters the function and/or expression of a gene that was at the site of the targeted chromosomal location. A transgenic “knock-in” animal is an animal generated from a mammalian cell that carries a genetic modification resulting from the insertion of a DNA construct targeted to a predetermined, specific chromosomal location that does not alter the function and/or expression of the gene at the site of the targeted chromosomal location. In both cases, the DNA construct may encode a reporter protein such as lacZ, protein tags, and proteins, including recombinases such as Cre and FLP.

General Description

This invention is based in part on elucidation of the coding sequence and function of the human receptor designated herein as Calcium Independent Receptor of Latrotoxin 3-Like receptor (CIRL3-L). The experiments described below identify the function of CIRL3-L as involved in the mediation of anxiety and anxiety-related motor activity, the regulation of psychiatric disorders such as schizophrenia and anxiety disorders, and the modulation of motor activity. Additionally, CIRL3-L may be involved in the mediation of seizures and related disorders. Accordingly, these discoveries provide new methods identifying agents useful for the therapeutic treatment of CIRL3-L-mediated conditions, such as anxiety disorders and seizures, by modulating CIRL3-L-activity. Further, the invention provides screening assays for identification of molecules capable of modulating CIRL3-L-mediated activity, e.g., physiological events affected by the activation or inhibition of CIRL3-L.

Protein and Nucleic Acid Sequence

Applicants have ascertained the amino acid sequence of a novel protein receptor designated herein as CIRL3-L (SEQ ID NO: 1). In addition, isolated nucleic acid sequences are described herein which encode this novel protein or portions thereof. Accordingly, the present invention includes the nucleic acid sequence designated herein as SEQ ID NO: 2, as well as nucleotide sequences that hybridizes under stringent conditions to the complement of the nucleotide sequence of SEQ ID NO:2 and which encodes CIRL3-L, wherein said stringent conditions are 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; and nucleotide sequences which, as a result of the degeneracy of the genetic code, differs from the nucleic acid of SEQ ID NO:2 or sequences which hybridize thereto and which encode CIRL3-L. Further encompassed by the invention are nucleic acids encoding a protein having 90%, 95%, or 99% identify to the protein of SEQ ID NO: 1. The similarity between different molecules can be expressed by the degree of homology between the nucleic acid or protein sequences. 50% homology means, for example, that 50 out of 100 nucleotides or amino acid positions in the sequences correspond to each other. The homology of proteins is determined by sequence analysis. Homologous DNA sequences can also be identified by the hybridization technique.

In addition, the invention contemplates vectors that comprise CIRL3-L encoding sequences, wherein the nucleic acid molecule is operatively linked to an expression control sequence capable of directing its expression in a host cell. The invention further contemplates host-vector systems for the production of CIRL3-L, including bacterial, yeast, insect, amphibian or mammalian cells.

Screening Assays

The present invention provides methods for identifying agents (e.g., candidate compounds or test compounds) that are capable of activating or inhibiting CIRL3-L-mediated activity or expression (collectively: modulating agents). Agents identified through the screening method of the invention are potential therapeutics for use in decreasing anxiety, nervous or compulsive motor activity, perseverative behaviors, and/or regulating psychiatric abnormalities such as anxiety disorders and schizophrenia, or neurological disorders such as autism or Tourette's syndrome, as well as in the treatment of seizures and related disorders.

Examples of agents to be tested by the screening methods of the invention include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that bind CIRL3-L are identified in a cell-based assay system. In accordance with this embodiment, cells expressing a CIRL3-L protein or protein fragment are contacted with a candidate (or a control compound), and the ability of the candidate compound to bind CIRL3-L is determined.

The cell may be of prokaryotic origin (e.g., E. coil) or eukaryotic origin (e.g., yeast or mammalian). In specific embodiments, the cell is for example, a COS-7 cell, a 293 human embryonic kidney cell, a NIH 3T3 cell, or Chinese hamster ovary (CHO) cell. Further, the cells may express a CIRL3-L protein or protein fragment endogenously or be genetically engineered to express a CIRL3-L protein or protein fragment. To identify ligands of CIRL3-L, cells expressing the receptor may be screened against a panel of know peptides utilizing a bioluminescent signal such as the aequorin luminescence assays (see, for example, Raddatz et al. (2000) J. Biol. Chem. 275:32452-32459 and Shan et al. (2000) J. Biol. Chem. 275:39482-39486, which publications are herein specifically incorporated by reference in their entireties). In these binding assays, the peptide to be tested is labeled. Cells expressing the CIRL3-L receptor are then incubated with labeled test compounds, in binding buffer, in cell culture dishes. To determine non-specific binding, unlabeled peptide may be added to the wells. After the incubation, bound and free peptides are separated and detection activity measured in each well.

The ability of the candidate compound to alter the activity of CIRL3-L can be determined by methods known to those of skill in the art, for example, by flow cytometry, a scintillation assay, immunoprecipitation or western blot analysis. For example, modulators of CIRL3-L-mediated activity may be identified using a biological readout in cells expressing a CIRL3-L protein or protein fragment. Agonists or antagonists are identified by incubating cells or cell fragments expressing CIRL3-L with test compound and measuring a biological response in these cells and in parallel cells or cell fragments not expressing CIRL3-L. An increased biological response in the cells or cell fragments expressing CIRL3-L compared to the parallel cells or cell fragments indicates the presence of an agonist in the test sample, whereas a decreased biological response indicates an antagonist.

In more specific embodiments, detection of binding and/or modulation of a test agent to a CIRL3-L protein may be accomplished by detecting a biological response, such as, for example, measuring Ca²⁺ ion flux, cAMP, IP₃, PIP₃ and transcription of reporter genes. Suitable reporter genes include endogenous genes as well as exogenous genes that are introduced into a cell by any of the standard methods familiar to the skilled artisan, such as transfection, electroporation, lipofection and viral infection. The invention further includes other end point assays to identify compounds that modulate (stimulate or inhibit) receptor activity, such as those associated with signal transduction.

The invention further provides a method of identifying an agent capable of modulating the expression of CIRL3-L, comprising (a) contacting a first population of cells expressing CIRL3-L with a candidate agent, (b) contacting a second population of cells expressing CIRL3-L with a control agent, and (c) comparing the level of CIRL3-L in the first and second populations of cells. In one embodiment, the level of CIRL-3L is greater in the first population of cells than in the second population of cells. In another embodiment, the level of CIRL3-L is less in the first population of cells than in the second population of cells. In a more specific embodiment, the level of CIRL3-L is determined by measurement of the corresponding mRNA.

In another embodiment, agents that bind CIRL3-L are identified in a cell-free assay system. In accordance with this embodiment, a CIRL3-L protein or protein fragment is contacted with a test (or control) compound and the ability of the test compound to bind CIRL3-L is determined. In vitro binding assays employ a mixture of components including a CIRL3-L protein or protein fragment, which may be part of a fusion product with another peptide or polypeptide, e.g., a tag for detection or anchoring, and a sample suspected of containing a natural CIRL3-L binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature that facilitates optimal binding. The mixture is incubated under conditions whereby the CIRL3-L protein binds the test compound. Incubation periods are chosen for optimal binding but are also minimized to facilitate rapid, high-throughput screening.

After incubation, the binding between the CIRL3-L protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radioactive emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that modulate (i.e., upregulate or downregulate) CIRL3-L-mediated activity or expression are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the CIRL3-L-mediated activity or expression is determined. Specifically, this method may be used to identify an agent capable of inhibiting anxiety, anxiety-related motor-activity, as well as nervous, compulsive motor activity, or to identify agents capable of modulating CIRL3-L-mediated psychiatric disorders and diseases. In addition, this method may specifically be used to identify an agent capable of treating seizures and related disorders.

Antibodies to Human CIRL3-L Protein and Ligands

The present invention provides for an antibody that specifically binds human CIRL3-L and is useful activating or inhibiting CIRL3-L-mediated activity. According to the invention, a CIRL3-L protein, protein fragment, derivative or variant, may be used as an immunogen to generate immunospecific antibodies. Such immunogens can be isolated by any convenient means, including the methods described above. Antibodies may be blocking antibodies or activating antibodies and include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA ) or subclass of immunoglobulin molecule.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105 ). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention that will be effective in the treatment of a CIRL3-L-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Therapeutic Methods and Combination Therapies

The invention is directed to therapeutically useful methods for treating any disease or condition which is improved, ameliorated, inhibited or prevented by modulation of CIRL3-L. Generally, inhibition of CIRL3-L may be useful to alleviate obsessive compulsive disorders. Activation of CIRL3-L is believed to be therapeutically useful in the treatment of autism or pervasive developmental disorders. In numerous embodiments, an agonist or antagonist of CIRL3-L may be administered in combination with one or more additional compounds or therapies.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a transgenic knock-out animal having a modified endogenous CIRL3-L gene. A transgenic animal can be produced by introducing nucleic acid into the male pronuclei of a fertilized oocyte, e.g., by microinjection, retroviral infection, and allowing the oocyte to develop in a pseudopregnant female foster animal. Still further, the invention contemplates a transgenic animal having an exogenous CIRL3-L gene generated by introduction of any CIRL3-L-encoding nucleotide sequence that can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the CIRL3-L protein to particular cells.

Transgenic animals containing a modified CIRL3-L gene as described herein are useful to identify CIRL3-L function. Further, animals containing an exogenous CIRL3-L gene, e.g., a human CIRL3-L gene, may be useful in an in vivo context since various physiological factors that are present in vivo and that could effect ligand binding, CIRL3-L activation, and signal transduction, may not be evident from in vitro cell-free or cell-based assays. Accordingly, it is useful to provide non-human transgenic animals to assay in vivo CIRL3-L protein function, including ligand interaction, the effect of specific mutant CIRL3-L proteins on CIRL3-L protein function and ligand interaction, and the effect of chimeric CIRL3-L proteins. It is also possible to assess the effect of null mutations, that is, mutations that substantially or completely eliminate one or more CIRL3-L protein functions.

Transgenic animals containing a modified CIRL3-L gene as described herein are useful as animal models of anxiety-related disorders, obsessive compulsive behavior or related disorders. The methods of using this model to screen for agents capable of reducing, ameliorating and/or inhibiting psychiatric disorders, motor activity, perseverative or compulsive behaviors, and anxiety, comprises administering a test agent to the animal and determining the ability of the test agent to reduce anxiety or anxiety-related motor activity, or modulate motor activity. The effectiveness of the test agent may be determined by behavioral observation, as described below.

Transgenic animals containing a modified CIRL3-L gene as described herein are also useful as non-human animal models of seizure and disorders associated therewith. The invention includes methods of using this model to screen for agents capable of treating of seizure and related disorders, comprising administering a test agent to the animal and determining the ability of the test agent to treat seizure and disorders associated therewith. The effectiveness of the test agent may be determined by seizure threshold, seizure severity, EEG amplitude or frequency changes, or histopathological evidence of seizure-related neural alterations including, but not limited to, neuronal cell death, mossy fiber sprouting, or gliosis.

Identification of Psychiatric Disorders Associated with CIRL3-L

A standard test for anxiety in mice is the elevated plus maze. In these experiments, a modified maze, containing only 2, rather than 4, arms was used. This maze was called the “Hemi-Maze”, because it represents “half” of the elevated plus maze. The “Hemi-Maze” assesses anxiety using the same principle upon which both the elevated plus maze and the light-dark exploration test are based. That is, rodents have two conflicting drives when placed into a novel environment. Rodents are driven to ensure their own safety by remaining in dark, sheltered regions of a novel environment, but are also driven to fully explore their environment, even if that exploration brings them into open, exposed areas of the environment. These mazes have two types of regions, one that is dark and more enclosed, and one that is light and more open. Animals with less anxiety will spend more time in the open regions, and those with greater anxiety will spend more time in the closed regions. The “Hemi-Maze” has two arms, one of which is open and exposed, with sides made of clear Plexiglas, and one of which is more enclosed, with a dark floor and sides painted black. In addition to measuring the proportion of time spent in each side, there are various other measures that can be taken to evaluate different types of anxiety. For instance, time to first cross can be used as a surrogate measure of “freezing,” a rodent behavior undertaken during extreme anxiety. In addition, defecations are counted, because animals tend to defecate more when anxious. Numbers of rears are measured, because anxious animals are hesitant to rear, an action that fully exposes their vulnerable abdomen. In the first couple of minutes in a novel environment, animals will not groom unless engaging in “nervous” motor behavior or compulsive stereotypic motor programs. Therefore, the presence of grooming in the first 2 minutes of the trial is recorded. Finally, the number of crosses is counted, as this can serve both as a measure of general activity, and also as a measure of exploratory drive.

Early Grooming. Early grooming is a sign of “nervous” motor activity or compulsive motor activity, such as that observed in anxiety disorders, such as obsessive-compulsive disorder. In obsessive-compulsive disorder the thalamus, cingulate cortex, and striatum have been heavily implicated, consistent with the distribution of CIRL3-Like by LacZ. CIRL3-Like null mutants show clear indications of the expression of anxious behaviors including increased guarding (fewer rears) and increased expression of anxiety-related stereotypic motor behaviors. CIR3L appears be involved in anxiety disorders such as obsessive-compulsive disorder.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Identification of Human CIRL3-L

CIRL3-Like is a receptor identified from genomic DNA. After bioinformatics refinement, the full-length receptor was confirmed by RT-PCR and sequencing as described below.

An extensive database (>4000 sequences) of all known GPCR protein sequences was compiled. The database was expanded by several rounds of homology search, BLASTp BLAST 2.0 was obtained from the NCBI ftp site (ftp://ncbi.nlm.nih.gov/blast/executables). This homology search was performed against public protein sequences from GenBank. The positions of putative transmembrane segments were annotated for each family member using a combination of homology (matching transmembrane positions to those of the closest homologue), hydrophobicity and alignment of key conserved residues to general models (Baldwin et al. (1997) J. Mol. Biol. 272:144-64). In addition to BLAST search, the CLUSTALW algorithm (CLUSTALW 1.7, Nucleic Acids Research, 22(22):4673-4680), which was downloaded from www.csc.fi/molbio/progs/clustalw/clustalw.html), was also used in some cases to align sequences for annotation of transmembrane regions.

New GPCR homologues were identified from human genomic DNA sequence as follows: Both finished and unfinished high throughput human genomic DNA sequence was downloaded weekly from the NCBI database which can be accessed via the internet. The DNA sequences were converted into predicted proteins using the GenScan program (Burge et al. (1997) J. Mol. Biol. 268:78-94 and (1998) Curr. Opin. Struct. Biol. 8:346-354). All proteins greater than 20 amino acids in length that were predicted using either optimal or suboptimal exons (see annotation of GenScan for a description of optimal and suboptimal exon prediction) (cutoff=0.1) were included. Each of the predicted proteins were compared using BLASTp to the GPCR database created as described supra using only those regions of each sequence in the GPCR database that extend from the first through the seventh transmembrane domain inclusive (Tms 1-7). Each predicted protein which showed homology to any member of the database (cutoff=e<10-4, e=expected value as defined by the BLAST program) was examined. Human CIR3L was initially identified as an open reading frame from a BAC clone. The identified intronless open reading frame was PCR-amplified from genomic. Then a PCR product was produced from cDNA derived from human placental RNA. The resulting PCR fragment was cloned into a series of expression vectors for functional analyses.

### Example 2

### Expression of Human CIRL3-L

CIRL3-Like was knocked out using VelociGene™ technology as described in U.S. patent application Ser. No. 09/732,234 filed 7 Dec. 2000, herein specifically incorporated by reference in its entirety. LacZ staining was performed on chimeras and mutant mice. CIRL3-Like is localized to many regions of the brain, with particularly dark expression in the thalamus and anterior cingulate cortex. These sites of expression suggest activities of this receptor in psychiatric disorders, with the anterior cingulate cortex showing striking functional abnormalities in many psychiatric disorders, such as in the anxiety disorders and schizophrenia. Specifically, abnormalities in FMRI have been shown in the anterior cingulate of patients with OCD, consistent with the expression pattern of CIRL3-Like.

### Example 3

### Behavioral Testing of CIRL3-L Knockouts

Two cohorts of F2 CIRL3L mice, all derived from the 157B-C10 clone, and one cohort of N2F2 mice, derived from the 157B-H6 clone, were selected for behavioral testing. All data shown are from the F2 mice, except for the addition of social interaction data from the N2F2 generation, containing a higher B57B1/6 genetic background. Both generations will be shown for social interaction because animals from both genetic backgrounds exhibited social impairments, but the nature of the impairment shifted with the shift of background. Both males and females were tested, although only wild types and knock-outs were included in the analyses (no heterozygote animals). Animals ranged in age from 2 to 6 months old and came from 9 separate litters. In the first cohort, 9 wild types were tested (4 females and 5 males) and 8 knock-outs were tested (3 females and 5 males). Analyses were conducted on 13 wild types (6 females and 7 males) and 13 knock-outs (6 females and 7 males) for the second cohort. The third, N2F2 cohort, contained 12 wild types (6 females and 6 males) and 13 knock-outs (8 females and 5 males). No significant differences were observed between genders for any measure. For each test, any animal with scores exceeding 2 standard deviations from the group mean were considered outlyers and were removed from the data set. For all behavioral tests, animals were acclimated to the testing room for 1 hour before testing. Animals were tested with the elevated plus maze (generalized anxiety), the light-dark exploration apparatus (generalized anxiety), the open field test (locomotor activity, compulsive motor anxiety, exploratory behavior), gait analysis (locomotor skills), the Morris water maze (learning and memory), social interaction on an open field (social skills), hot plate test (centrally-mediated acute thermal analgesia), tail flick test (spinally-mediated acute thermal analgesia), von Frey hairs (cutaneous sensation), visual cliff (visual depth perception), and the Y maze (perseverative tendencies). Results will not be shown (and no significant differences were detected) for the locomotor or exploratory elements of the open field, the Morris water maze, the visual cliff, or the Y maze.

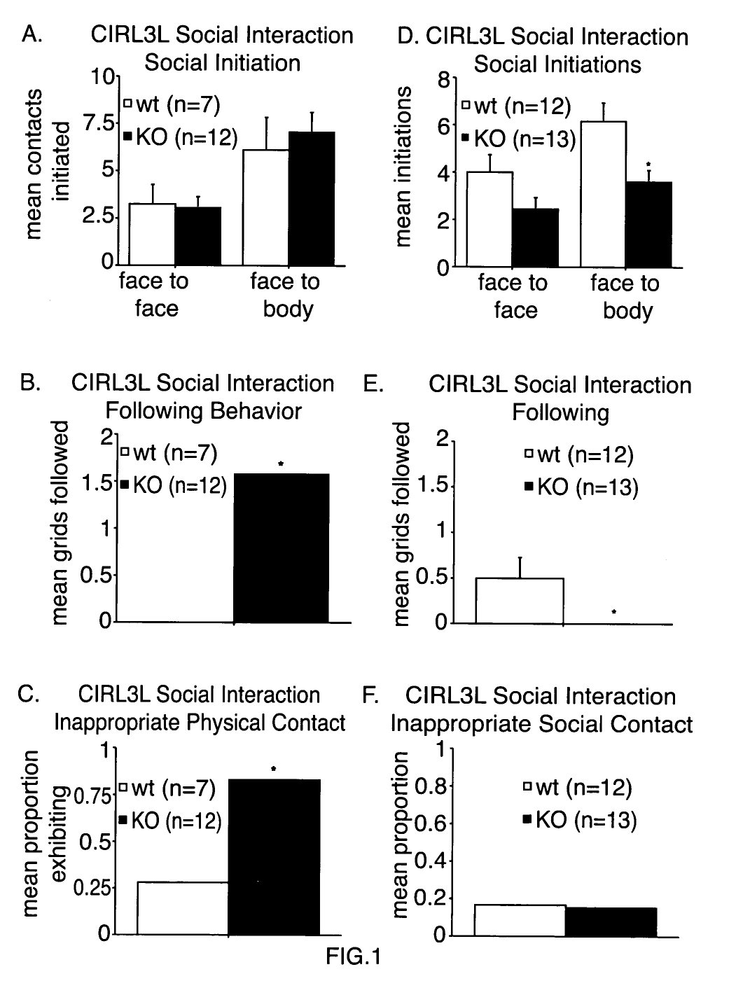

CIRL3-L mutant mice show significant social impairment, as measured by their response to a novel mouse of similar strain, age, and gender on a novel open field. Although the particular abnormality observed depended on the background of the mouse (that is, F2s or N2F2s), all mice showed these abnormalities. F2 mice showed no hesitation about initiating social contact with a novel mouse (FIG. 1A), but showed consistently abnormal patterns of mouse social interaction, including significant following (tracking) of the novel mouse (FIG. 1B) and inappropriate contact (such as walking over the top of the novel mouse; FIG. 1C). The N2F2 generation, which was more genetically similar to the C57B1/6 strain than the F2 generation (75% as opposed to 50%), showed more serious social impairments, characterized primarily by a reticence to engage in social contact (significantly reduced social initiation, FIG. 1D). Because of this reticence, the N2F2 mutants did not engage in significantly more following (FIG. 1E) or other inappropriate behaviors (FIG. 1F) but, rather, avoided contact with the novel mouse. Social impairments are hallmark characteristics of the pervasive developmental disorders and schizophrenia.

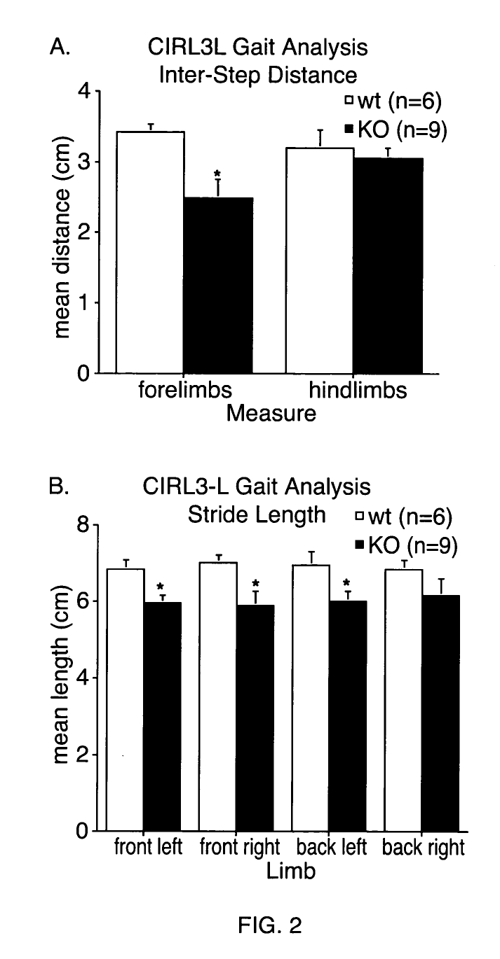

CIRL3-L KO mice also showed a significant gait abnormality, based on results on a footprint analysis. While their base of support and their toe spread was normal (data not shown), the KO mice showed significantly decreased stride length and forepaw inter-step distance than their wild-type littermates (FIG. 2A and FIG. 2B). These results suggest that the CIRL3-L KOs have either decreased muscle strength, abnormally increased muscle tone, or decreased balance/stability. Any of these consequences can result from primary muscle abnormalities or deficiencies in the neural motor systems. Mild gait abnormalities, such as those observed in these mice, can be observed in a wide range of human developmental disorders, including the pervasive developmental disorders, attention deficit hyperactivity disorder (ADHD), and schizophrenia.

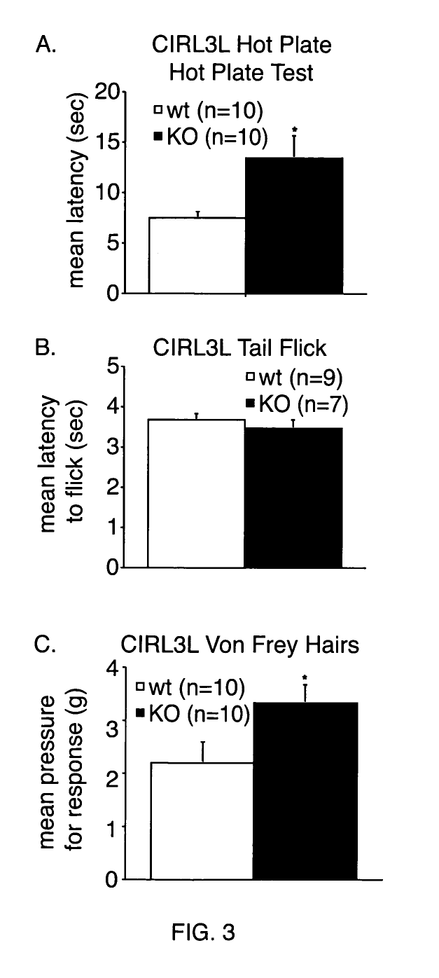

In addition to exhibiting motor abnormalities, CIRL3-L KO mice show a significantly decreased nociceptive response on the hot plate test of centrally-mediated acute thermal pain (FIG. 3A). In contrast, responses to spinally-mediated acute thermal pain are normal in CIRL3-L KOs, as measured by latency to withdrawal their tails in response to a painful stimulus on the tail flick test (FIG. 3B). Because the tail flick response is normal, it is possible that the CIRL3-like KO mice have poor central modulation of pain, which could include abnormalities either in general sensory processing/sensory gating mechanisms, or primary abnormalities in brain pain systems. The finding that CIRL3-L KO mice also show decreased sensitivity to cutaneous somatosensation (FIG. 3C) suggests that it is more likely that general sensory processing or sensory gating deficits might be present in these mice. Sensory gating abnormalities are common in schizophrenia, the pervasive developmental disorders (especially autism, Asperger's syndrome, and PDD-NOS), and in attention deficit-hyperactivity disorder (ADHD).

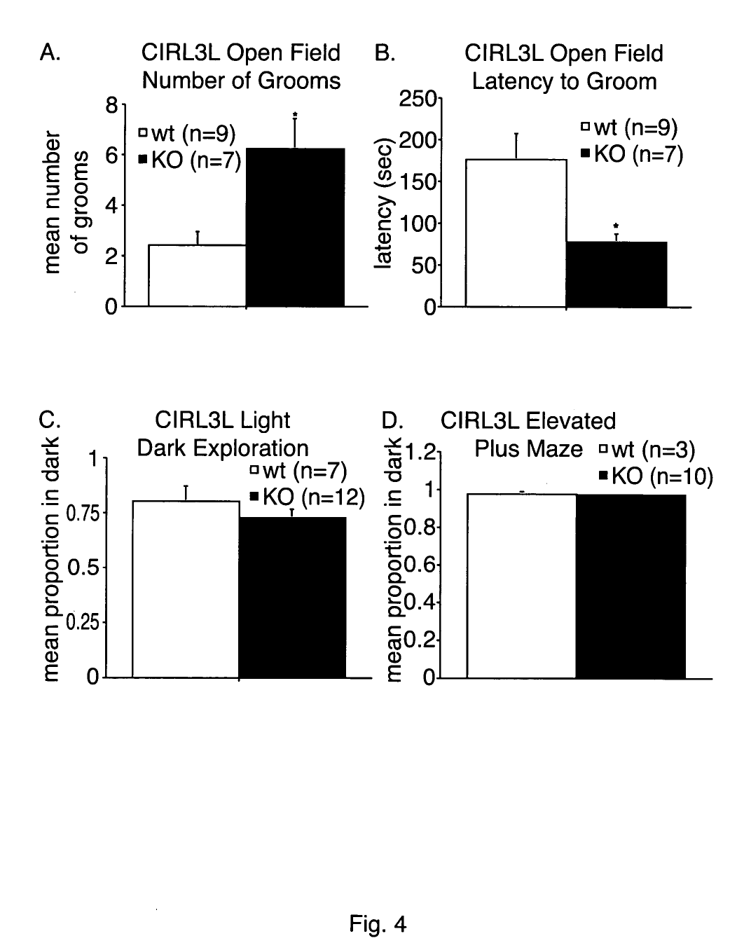

The CIRL3-L null KO mice showed significantly more grooming than their wild type littermates, suggesting an increase in compulsive motor anxiety (FIG. 4A). Consistent with this finding, the CIRL3-L null mutants also started grooming significantly earlier than the wild types, consistent with the increased tendency toward increased motor compulsions (FIG. 4B). In contrast, the KOs were no different than the wild types in either measure of generalized anxiety (light-dark exploration task, FIG. 4C or elevated plus maze, FIG. 4D). These findings suggest that the CIRL3-L KOs have a very specific increase in compulsive motor anxiety, in the absence of overt increases in generalized anxiety. This profile of abnormal compulsive motor anxiety in the absence of overt generalized anxiety can be reported in obsessive compulsive disorder (OCD) and the pervasive developmental disorders, especially autism and Asperger's Syndrome.

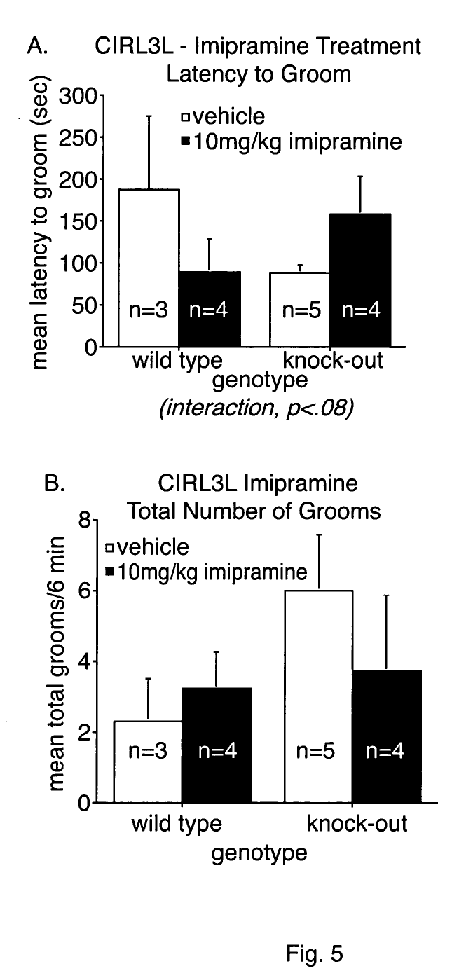

Because motor compulsions are treated with tricyclics or selective serotonin reuptake inhibitors (SSRIs), a small pilot study was conducted to treat a group of CIRL3-L KO mice with imipramine, a tricyclic drug commonly used for this purpose. Imipramine tended to decrease the number of grooms in the CIRL3-L mutants compared to mutants treated with vehicle (FIG. 5A), as well as increase the latency to groom in these animals (FIG. 5B). Wild type animals, in contrast, were agitated by the drug, and tended to show earlier grooming than they would normally have shown. Imipramine is commonly used in the treatment of OCD, and is sometimes used in the pervasive developmental disorders, although its use is limited by its cardiac side effects.

What is claimed is:

1. A method of identifying an agent capable of modulating a Calcium Independent Receptor of Latrotoxin 3-Like (CIRL3-L) protein activity or expression, comprising
(a) contacting a test agent with a CIRL3-L protein; and
(b) determining the ability of the test agent to modulate CIRL3-L activity or expression.

2. The method of claim 1, wherein the agent is an inhibitor of CIRL3-L activity or expression.

3. The method of claim 2, wherein the agent is an inhibitor of CIRL3-L expression.

4. The method of claim 3, wherein the agent is a CIRL3-L antisense molecule.

5. The method of claim 3, wherein the agent is a siRNA molecule capable of interfering with the expression of the gene encoding CIRL3-L.

6. The method of claim 2, wherein the agent is an inhibitor of CIRL3-L activity.

7. The method of claim 6, wherein the agent is a blocking antibody.

8. The method of claim 2, wherein the agent is an activator of CIRL3-L activity or expression.

9. The method of claim 8, wherein the agent is an activator of CIRL3-L expression.

10. The method of claim 9, wherein the agent is an activating antibody.

11. An in vivo method for identifying an agent capable of modulating a Calcium Independent Receptor of Latrotoxin 3-Like (CIRL3-L) protein, comprising:
(a) administering a test agent to an animal expressing a CIRL3-L protein; and
(b) determining the ability of the test agent to modulate CIRL3-L.

12. The method of claim 11, wherein the ability of the test agent to modulate CIRL3-L is determined by measuring behavior of psychiatric disorders.

13. The method of claim 12, wherein the psychiatric disorders are measured by a test selected from the group consisting of elevated plus maze, open field testing, light-dark exploration tests, tests of social interation, sensory tests, sensory gating tests, quantification of animal freezing, defecations, rears and early grooming.

14. A non-human animal comprising an altered or deleted Calcium Independent Receptor of Latrotoxin 3-Like (CIRL3-L) gene.

15. The non-human animal of claim 16, characterized by exhibiting anxiety-related disorders, obsessive, perseverative, or compulsive behavior or related disorders.

16. The non-human animal of claim 14, characterized by exhibiting pervasive developmental disorders.

17. The non-human animal of claim 14, selected from the group consisting of a mouse, a rat, a rabbit, a guinea pig, a hamster, a cat, a dog, and a sheep.


SEQUENCE LISTING (4 sequences)

SEQ ID NO: 1 - protein, 348 aa, Homo sapien
MGKISSLPTQLFKCCFCDFLKVKMHTMSSSHLFYLALCLLTFTSSATAGPRTLCGAELVDALQFVCGDRGFYFNKPTGYGSSSRRAPQTGIVDECCFRSCDLRRLEMYCAPLKPAKSAGTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 421 aa, Homo sapien
MGKISSLPTQLFKCCFCDFLKVKMHTMSSSHLFYLALCLLTFTSSATAGPRTLCGAELVDALQFVCGDRGFYFNKPTGYGSSSRRAPQTGIVDECCFRSCDLRRLEMYCAPLKPAKSAGTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGTGGPRTLCGAELVDALQFVCGDRGFYFNKPTGYGSSSRRAPQTGIVDECCFRSCDLRRLEMYCAPLKPAKSA

SEQ ID NO: 3 - protein, 321 aa, Homo sapien
MGIPMGKSMLVLLTFLAFASCCIAAYRPSETLCGGELVDTLQFVCGDRGFYFSRPASRVSRRSRGIVEECCFRSCDLALLETYCATPAKSEGTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 391 aa, Homo sapien
MGIPMGKSMLVLLTFLAFASCCIAAYRPSETLCGGELVDTLQFVCGDRGFYFSRPASRVSRRSRGIVEECCFRSCDLALLETYCATPAKSEGTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGTGAYRPSETLCGGELVDTLQFVCGDRGFYFSRPASRVSRRSRGIVEECCFRSCDLALLETYCATPAKSE